(19) United States
(12) Patent Application Publication
(10) Pub. No.: US 2022/0072798 A1
(43) Pub. Date: Mar. 10, 2022

(54) Systems And Methods For Forming A Customized Therapeutic Support

(71) Applicant: Matheny Enterprises, LLC, Plano, TX (US)

(72) Inventor: Keith E. MATHENY, Plano, TX (US)

(21) Appl. No.: 17/416,030

(22) PCT Filed: Feb. 21, 2020
(86) PCT No.: PCT/US2020/019185

§ 371 Date: Jun. 18, 2021

Related U.S. Application Data
(60) Provisional application No. 62/784,122, filed on Dec. 21, 2018.

(51) Int. Cl. B29C 64/393; C12Q 1/689; B33Y 10/00; B33Y 50/02; B33Y 80/00

(52) U.S. Cl. CPC B29C 64/393; C12Q 1/689; B29L 2031/753; B33Y 50/02; B33Y 80/00; B33Y 10/00

(57) ABSTRACT

A method of providing therapy to a tissue site. The method may include collecting three-dimensional (3-D) data associated with the tissue site, selecting at least one parameter of a customized tissue support based upon the 3-D data, and fabricating the customized tissue support including the design parameter. The method may further include collecting microbiome data associated with the tissue site. Collecting microbiome data may include collecting a biological sample. Collecting microbiome data may further include amplifying genetic material from the biological sample. Collecting the microbiome data may further include evaluating the genetic material from the biological sample. In some embodiments, the method may further include selecting at least one parameter of a customized splint based upon the microbiome data.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Therapeutic medical supports, such as implants, splints, and stents, are commonly employed to provide various therapies, for example, to replace missing biological tissue, support damaged biological tissue, enhance a tissue site, or correct abnormalities at a tissue site. However, many conventional, currently-used therapeutic supports just narrowly tip the balance in favor of therapeutic benefit when weighed against the risks and side effects, either at the intended site of treatment or elsewhere within the body. Additionally, many current therapeutic devices may be beneficial for only finite periods of time, but they may outlast their usefulness within the body, and become potentially harmful the longer they remain within the patient.

As such, there is a need for improved means of medical therapies and/or therapeutic devices that, for example, will allow therapeutics to be delivered locally, while subsisting for a desired, beneficial duration of time.

### SUMMARY

Disclosed herein in a method of providing therapy to a tissue site. In some embodiments, the method comprises collecting three-dimensional (3-D) data associated with the tissue site. The method may also comprise selecting at least one parameter of a customized tissue support based upon the 3-D data. The method may also comprise fabricating the customized tissue support including the design parameter.

In some embodiments, the method further comprises collecting microbiome data associated with the tissue site. Collecting microbiome data may include collecting a biological sample. Collecting microbiome data may further include amplifying genetic material from the biological sample. Collecting the microbiome data may further include evaluating the genetic material from the biological sample. In some embodiments, the method may further include selecting at least one parameter of a customized splint based upon the microbiome data.

### BRIEF DESCRIPTION OF THE DRAWINGS

For a more complete understanding of the present disclosure and the advantages thereof, reference is now made to the following brief description, taken in connection with the accompanying drawings and detailed description:

FIG. 1 is a diagrammatic representation of an embodiment of a system for providing a customized tissue support.

FIG. 2 is a diagrammatic representation of an embodiment of the customized tissue support modeling system.

FIG. 3 is a diagrammatic representation of an embodiment of a method for providing a customized tissue support.

### DETAILED DESCRIPTION OF THE EMBODIMENTS

In the drawings and description that follow, like parts are typically marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness. The present invention is susceptible to embodiments of different forms. Specific embodiments are described in detail and are shown in the drawings, with the understanding that the present disclosure is not intended to limit the invention to the embodiments illustrated and described herein. It is to be fully recognized that the different teachings of the embodiments discussed herein may be employed separately or in any suitable combination to produce desired results.

Disclosed herein are embodiments of systems and methods for providing a customized therapeutic support (referred to herein as a “CTS”), and methods for providing therapy using a CTS. As used herein, a “customized” therapeutic support is intended to refer to a therapeutic support having at least one parameter that has been selected on the basis of a particular tissue site of a particular patient for whose use the CTS is intended.

In various embodiments, the tissue site may include a bodily cavity, a lumen, a vessel, or duct. For example, the tissue site may include at least a portion of the cranial cavity; at least a portion of the spinal cavity; at least a portion of the thoracic cavity; at least a portion of the abdominal cavity; at least a portion of the pelvic cavity; a blood vessel, vein, or artery; a lymphatic vessel, node, or duct; at least a portion of the gastrointestinal tract; at least a portion of the upper respiratory tract, such as the nasal cavity and paranasal sinuses, pharynx, larynx; at least a portion of the lower respiratory tract, such as the trachea; or at least a portion of the reproductive, urinary, and/or genital tracts, such as the uterus and fallopian tubes.

In various embodiments, the CTS may comprise and/or be configured as an implant, a stent, a splint, a scaffold, or combinations thereof. The CTS may be configured to perform any suitable therapeutic function or combination of therapeutic functions when deployed with respect to a patient, particularly, when deployed with respect to the tissue site. For example, the CTS may be configured to replace missing biological tissue; to maintain damaged biological tissue in a particular orientation or placement; to correct deformed biological tissue; to maintain the patency of a passageway, cavity, or orifice; to constrict a passageway, cavity, or orifice; to inhibit constriction of a passageway, cavity, or orifice; to promote constriction of a passageway, cavity, or orifice; to maintain ventilation or free exchange of fluids into and/or out of a physiologic chamber; to physically impede access to a physiologic chamber; or combinations thereof. In some embodiments, the CTS may be configured to apply a force with respect to a tissue, for example, a force sufficient to displace the tissue toward a desired position and/or a force sufficient to maintain the tissue at a desired position.

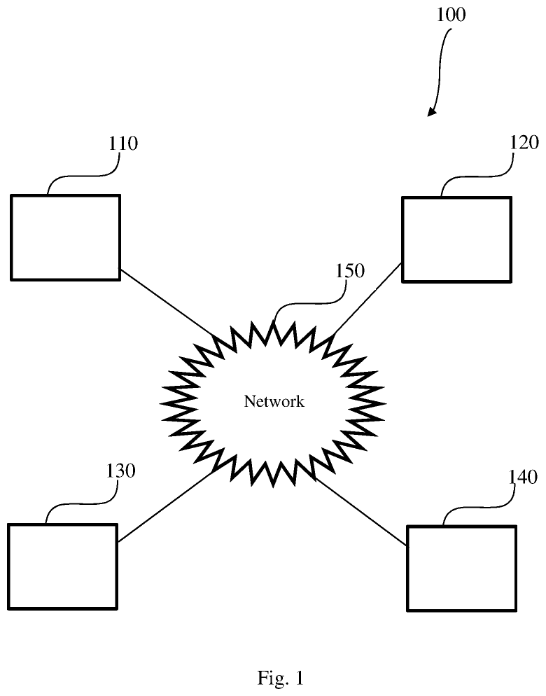

In some embodiments, the disclosed systems for providing a CTS may generally include one or more devices configured to image the tissue site, a computing system generally configured to generate a model representing the CTS, and a fabrication unit generally configured to produce the CTS according to the model representing the CTS. Referring to FIG. 1, a diagrammatic representation of an embodiment of a system 100 for providing a CTS is shown. In the embodiment of FIG. 1, the system 100 comprises microbiome diagnostic equipment 110, a medical imaging device 120, a CTS modeling system 130, and a fabrication unit 140.

In some embodiments, the microbiome diagnostic equipment 110, may be generally configured to collect or sample microbiome, for example, a pathogenic or infectious agent, at, associated with, or proximate to the tissue site. For example, in some embodiments, the microbiome diagnostic equipment 110 may comprise a tissue sampling or collection device, such as a buccal swab, a syringe (e.g., to draw blood or plasma), a blade, scalpel, or punch (e.g., to excise a biopsy), or the like.

Additionally or alternatively, in some embodiments, the microbiome diagnostic equipment 110, may be generally configured to amplify, identify, classify, quantify, or otherwise assess the microbiome collected or sampled from the tissue site. For example, in some embodiments the microbiome diagnostic equipment 110 may include a thermal cycler, for example, suitable in the performance of a polymerase chain reaction (PCR). Additionally or alternatively, the microbiome diagnostic equipment 110 may include one or more bioassay kits, suitable to identify one or more microorganisms and/or to ascertain the presence or likelihood of infection by one or more microorganisms. In various embodiments, the bioassay kit may be, for example, a DNA-based assay, a RNA-based assay, a protein-based assay, a cell-count or cell proliferative assay, or the like.

In some embodiments, the medical imaging device 120 may be configured to provide one or more images of the tissue site. For example, in some embodiments, the medical imaging device 120 may be configured to perform one or more scans of a tissue site, for example, so as to produce one or more images of the tissue site. In some embodiments, the one or more images may include a composite image, for example, a plurality images (e.g., slices) that, taken together, provide a three-dimensional representation of the tissue site. In some embodiments, the medical imaging device 120 may include a X-ray machine, an magnetic resonance imaging (MRI), an ultrasound, a X-ray computed tomography (CT), photoacoustic imaging device, tactile imaging device, a Positron-emission tomography (PET) device, or combinations thereof.

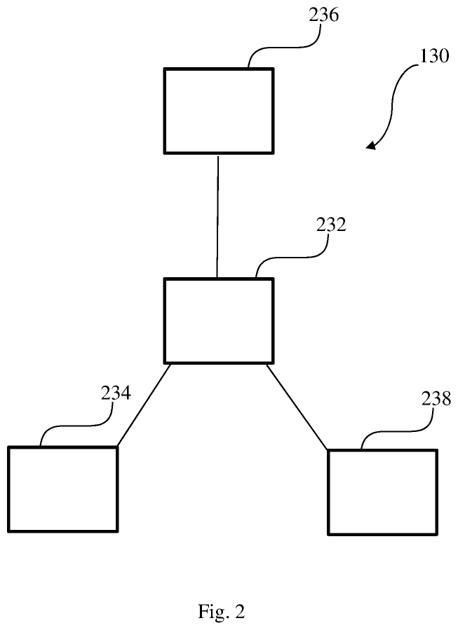

In some embodiments, the CTS modeling system 130 may comprise a computing system configured to operate one or more software programs that, when operated, may be effective to generate a model representing the CTS, for example, based upon one or more images of the tissue site. For example, referring to FIG. 2, a diagrammatic representation of an embodiment of the CTS modeling system 130 is illustrated. In some embodiments, the CTS modeling system 130 may comprise or be characterized as a computer. For example, in the embodiment of FIG. 2, the CTS modeling system 130 includes a processor 232 configured to execute one or more one or more modules including a CTS modeling module 234, memory 236, and a communication interface 238.

In some embodiments, the CTS modeling module 234 may be executed by the processor to generate a model representing the CTS based on the one or more images of the tissue site from the medical imaging device 120. For example, CTS modeling module 234 may be any suitably-configured application, program, module, process, or other software that utilizes the one or more images of the tissue site from the medical imaging device 120 and/or data obtained from these images of the tissue site to generate a model representing the CTS. Regardless of the particular implementation, “software” may include software, firmware, wired or programmed hardware, or any combination thereof as appropriate. For example, the CTS modeling module may be written or described in any appropriate computer language including C, C++, Java, Visual Basic, assembler, Perl, any suitable version of 4GL, as well as others. It will be understood that while CTS modeling module 234 is illustrated in FIG. 1 as a single module, the CTS modeling module 234 may include numerous other sub-modules or may instead be a single multi-tasked module that implements the various features and functionality through various objects, methods, or other processes. Further, while illustrated as internal to CTS modeling system 130, one or more processes associated with CTS modeling module 234 may be stored, referenced, or executed remotely. For example, a portion of CTS modeling module 234 may be a web service that is remotely called, while another portion of CTS modeling module 234 may be an interface object bundled for processing at a remote client. Moreover, the CTS modeling module 234 may be a child or sub-module of another software module or enterprise application (not illustrated) without departing from the scope of this disclosure. In some embodiments, the CTS modeling module 234 may be comprise a plug-in, add-in, add-on, or extension suitable to modify the operation of a suitable computer-aided design (CAD) program, examples of which include AutoCAD and Inventor available from Autodesk, and Solidworks available from Dassault.

In some embodiments, the processor 232 is, for example, a central processing unit (CPU), a blade, an application specific integrated circuit (ASIC), or a field-programmable gate array (FPGA). Although FIG. 2 illustrates a single processor 232 in CTS modeling system 130, in other embodiments multiple processors 232 may be used according to particular needs and reference to processor 232 is meant to include multiple processors 232 where applicable. In the illustrated embodiment, processor 232 executes CTS modeling module 234 as well as other modules, as necessary. For example, in some embodiments, the processor 232 may execute software that manages or otherwise controls the operation of the fabrication unit 140 during a fabrication of the CTS.

In some embodiments, the memory 236 is communicably coupled to the processor 232 and may include any memory or database module and may take the form of volatile or non-volatile memory including, without limitation, magnetic media, optical media, random access memory (RAM), read-only memory (ROM), removable media, or any other suitable local or remote memory component. The memory 236 may include any signal or data necessary to utilize the one or more images of the tissue site and/or data obtained from these images of the tissue site to generate a model representing the CTS. The memory may also include other appropriate data such as VPN applications or services, firewall policies, a security or access log, print or other reporting files, HTML files or templates, data classes or object interfaces, child software applications or sub-systems, and others.

In some embodiments, the communication interface 238 may facilitate communication between CTS modeling system 130 and various other devices, such as the medical imaging device 120, the fabrication unit 140, or other computing systems and devices. The interface 238 may comprise logic encoded in software and/or hardware in a suitable combination and operable to allow the CTS modeling system 130 to communicate, for example, via a network. For example, the interface 238 may comprise software supporting one or more communications protocols or hardware operable to communicate physical signals.

In some embodiments, the CTS modeling system 130 may further comprise one or more peripherals communicably coupled to and/or integrated with the CTS modeling system 130. In some embodiments, the peripherals may include a user interface, such a display devices (e.g., a LCD, a CRT, other display screen), one or more data input devices (e.g., keyboard, mouse, light pin, a touchscreen, or otherwise); one or more data storage devices (e.g., CD-ROM, DVD, flash memory, or otherwise); other input/output (I/O) devices; and combinations thereof.

In some embodiments, the fabrication unit 140 may be configured to fabricate the CTS, for example, based upon the model representing the CTS generated by the CTS modeling system 130. In some embodiments, the fabrication unit 140 may operate based upon additive manufacturing principles, subtractive manufacturing principles, or combinations thereof. For example, in some embodiments the fabrication unit 140 may comprise a suitable stereolithography device, for example, a 3-D printer configured for optical fabrication, photo-solidification, or resin printing, solid ground curing (SGC) or combinations thereof. Additionally or alternatively, the fabrication unit may comprise a laminated object manufacturing (LOM) unit, a selective laser sintering (SLS) unit, a fused deposition modeling unit, a powder printer, or combinations thereof. Additionally or alternatively, the fabrication unit 140 may comprise a computer numerical control (CNC) mill (e.g., a CNC router), a laser cutting unit, or combinations thereof. Additionally or alternatively, in some embodiments the fabrication unit 140 may include an injection molding machine (e.g., an injection press).

In the embodiment of FIG. 1, the microbiome diagnostic equipment 110, the medical imaging device 120, the CTS modeling system 130, and the fabrication unit 140 may be configured communicate with various other components of the system 100. For example, in the embodiment of FIG. 1, the microbiome diagnostic equipment 110, the medical imaging device 120, the CTS modeling system 130, and the fabrication unit 140 being in signal communication via a network 150. The network 150 may be configured to facilitate wireless or wired communication between two or more components. The network 150 may be all or a portion of an enterprise or secured network. While illustrated as a single or continuous network, network 150 may be logically divided into various sub-nets or virtual networks without departing from the scope of this disclosure. The network 150 may communicate, for example, via Internet Protocol (IP) packets, Frame Relay frames, Asynchronous Transfer Mode (ATM) cells, voice, video, data, and other suitable information between network addresses. The network 150 may include one or more local area networks (LANs), radio access networks (RANs), metropolitan area networks (MANs), wide area networks (WANs), all or a portion of the global computer network known as the Internet, and/or any other communication system or systems at one or more locations.

While the embodiment of FIG. 1 illustrates the components of the system 100 communicating via the network 150, additionally or alternatively, data may be communicated between any two components via any suitable medium or communication protocol. For example, in some embodiments, the signal communication need not be a physical connection between any two components. For example, data may be communicated via between two or more components via a physical medium that is transferred between devices, for example, a CD-ROM, a DVD, a flash-drive, a or the like.

Additionally or alternatively, while the embodiment of FIG. 1 illustrates the components of the system 100 as distinct, in some embodiments at least a portion of one or more components of the system 100 and/or their functionalities may be integrated within or common to another component. For example, in some embodiments, the functionality disclosed herein as being associated with the CTS modeling system 130 may, in some embodiments, be incorporated within fabrication unit 140.

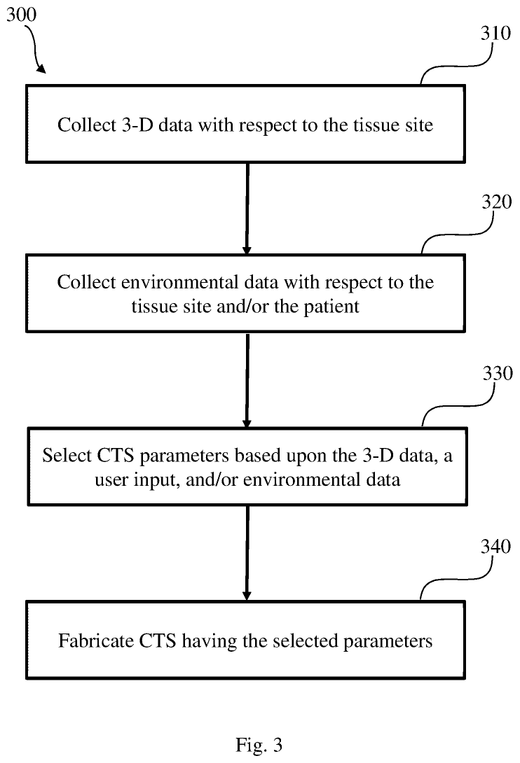

In some embodiments, the disclosed methods for providing a CTS may generally include the steps of characterizing a tissue site or a portion thereof, selecting parameters for the CTS on the basis of the characterization of the tissue site, and fabricating the CTS. Referring to FIG. 3, an embodiment of a method 300 for providing a CTS is illustrated diagrammatically.

In some embodiments, the method may include characterizing, for example, on the basis of the physical attributes of the tissue site, the biological attributes of the tissue site, or combinations thereof.

For example, in the embodiment of FIG. 3, the method 300 includes collecting 3-D data 310 with respect to the tissue site. In various embodiments, the 3-D data may include the size of tissue site and/or some portion of the tissue site; the physical dimensions of one or more features of the tissue site; the presence of an abnormality, deformity, or injury; the extent to which the tissue site is abnormal or deformed; or combinations thereof.

In some embodiments, collecting 3-D data 310 with respect to the tissue site may comprise imaging at least a portion of the tissue site. In various embodiments, any suitable imaging modality or combination of modalities may be employed to image the tissue site, examples of which may include, but are not limited to, X-ray, magnetic resonance imaging (MRI), ultrasound, X-ray computed tomography (CT), photoacoustic imaging, tactile imaging, positron-emission testing (PET) scans. Referring again to the embodiment of FIG. 1, in some embodiments the tissue site or a portion thereof may be imaged via the operation of the medical imaging device 120. Operation of the medical imaging device 120 may enable the size and/or proportions of one or more features of the tissue site to be determined, for example, the size of tissue site and/or some portion of the tissue site; the physical dimensions of one or more features of the tissue site; the presence of an abnormality, deformity, or injury; the extent to which the tissue site is abnormal or deformed; or combinations thereof.

Additionally or alternatively, in some embodiments collecting 3-D data 310 on the tissue site may also include generating one or more representations of the tissue site. For example, the imaging modality or combination of imaging modalities employed may yield one or more files including various data with respect to the tissue site. For example, in the embodiment of FIG. 1, one or more images may be generated via the operation of the medical imaging device 120. In various embodiments, the one or more representations of the tissue site may include data relating to the size of the tissue site and/or some portion of the tissue site; the physical dimensions of one or more features of the tissue site; the presence of an abnormality, deformity, or injury; the extent to which the tissue site is abnormal or deformed; or combinations thereof. For example, the one or more representations of the tissue site may include data relating to features of the tissue such as the size and/or shape of a cavity, or orifice; size and/or shape of a route of access (e.g., ingress or egress) with respect to the tissue site.

In some embodiments, the one or more representations may include a 3-D representation of the tissue site. In some embodiments, a representation of the tissue site may be viewable by a physician, although a representation need not be in a format that is necessarily viewable. In various embodiments, the one or more imaging modalities employed may perform a plurality of scans of the tissue site and the plurality of scans may be combined, for example, by computer, to produce a 3-D representation of the tissue site.

In some embodiments, the medical imaging device 120 may produce a plurality of segmented images. The image segments may serve as an input for modeling software configured to utilize the image segments to develop a 3-D model of the tissue site that was imaged. For example, taken cumulatively, the image segments yield 3-D data about the tissue site. In some embodiments, the one or more representations of the tissue site (for example, files) may be compliant with the Digital Imaging and Communication in Medicine (DICOM) Standard.

Additionally or alternatively, in some embodiments collecting 3-D data 310 on the tissue site may include performing a physical inspection. For example, a physician may visually inspect the tissue site. In some embodiments, the physician may create images representative of the tissue site on based upon such a visual inspection. Additionally or alternatively, in some embodiments the physician may correct, edit, confirm, or supplement a representation of the tissue site obtained by imaging the tissue site (e.g., via one or more imaging modalities) based upon a visual inspection of the tissue site.

In the embodiment of FIG. 3, the method 300 includes collecting environmental data 320 with respect to the tissue site and/or the patient. In various embodiments, the environmental data may include the presence of microbiome, for example, a pathogenic or infectious agent, at, associated with, or proximate the tissue site; the identity or classification of microbiome (e.g., a pathogen) present at or proximate to the tissue site, for example, a viral pathogen, a bacterial pathogen, a fungal pathogen, a prionic pathogen, a protozoal pathogen, a parasitic pathogen, or another potentially-disease-causing micro-organism; the pathogenicity (e.g., amount and/or activity) of any pathogens present at or near the tissue site; the pH of the tissue site; the presence of contaminants at the tissue site; the health of the tissue site; or combinations thereof.

In some embodiments, collecting environmental data 320 with respect to the tissue site and/or the patient may include obtaining a sample, for example, a buccal swab, a blood or plasma sample, a biopsy, a urine sample, a stool sample, or the like.

In some embodiments, the microorganisms in the sample may be amplified, such as by a polymerase chain reaction (PCR) or Rapid PCR. In some embodiments, the biological sample may be subjected to a suitable bioassay, for example, a DNA-based assay, a RNA-based assay, a protein-based assay, a cell-count or cell proliferation assay, or the like. For example, in some embodiments, the microbiome associated with the tissue site may be evaluated on the basis of genetic material (e.g., DNA and/or RNA) collected and amplified from the tissue site. In the embodiment of FIG. 1, various microbiome diagnostic equipment 110 may be utilized to collect the sample from the tissue site, to amplify the some or all of the microorganisms of the sample, to assay the microorganisms of the sample, or combinations thereof.

In various embodiments, one or more parameters of the CTS may be selected based on the characterization of the tissue site. For example, in various embodiments, one or more parameters of the CTS may be selected based upon the 3-D data associated with the tissue site, the microbiological data associated with the tissue site, or both. Additionally, in some embodiments, one or more parameters may be selected further based upon one or more inputs from a user, such as a physician. A given parameter may be selected on the basis of multiple factors and inputs. Examples of parameters of the CTS that may be selected include, but are not limited to, the dimensions of the CTS; the shape, design, and/or configuration of the CTS, the predicted mechanical activity exhibited by the CTS upon being deployed at the tissue site; the composition of the CTS, including one or more active agents; the duration over which the CTS is intended to be deployed at the tissue site; the duration over which the CTS is intended to degrade; and combinations thereof.

In the embodiment of FIG. 3, the method 300 includes selecting a parameter of the CTS based upon the 3-D data, based upon a user input, based upon the environmental data, or combinations thereof 330. For example, in various embodiments, the parameters of the CTS may be selected such that the CTS may interact with the tissue site in a desired way or exhibit a desired characteristic when deployed with respect to the tissue site. For example, in various embodiments, one or more parameters of the CTS may be selected such that, when deployed with respect to the tissue site, the CTS is configured to fill a void-space (e.g., as packing); to support tissue growth (e.g., as scaffolding); to maintain patency of a passageway or lumen (e.g., as a stent or splint); to constrict a passageway or lumen; to exert mechanical activity with respect to the tissue site or a portion of the tissue site, for example, so as to expand one or more dimensions of the tissue site; to fully or partially degrade while present at the tissue site; to exhibit a desired biological activity at the tissue site, for example, so as to combat or retard a pathogen; to deliver an active material to the tissue site so as to promote healing, improve tissue health, or combat or retard a pathogen; or combinations thereof. In some embodiments, the selection of the parameter of the CTS based upon the 3-D data, the user input, and/or the environmental data may be selected via the implementation of one or more algorithms or protocols. For example, one or more of the 3-D data, the user input, and/or the environmental data may provide an indication of the tissue site intended to be treated, the particular size and dimensions of the tissue site, the presence of an abnormality at the tissue site, the presence of certain microbiome. Based upon such indicia, one or more algorithms or protocols may be employed to select one more of parameters of the CTS, for example, related to its size, shape, and/or composition, such that when the CTS is deployed with respect to the particular tissue site, the CTS may be effective to exhibit a desired characteristic when deployed with respect to the tissue site.

In some embodiments, by selecting one or more parameters of the CTS such that the CTS such that the CTS interacts with the tissue site in a desired way (e.g., by customizing the CTS with respect to a particular tissue site), the CTS may be effective to exhibit various particular, desired characteristics. For example, in various embodiments the dimensions/size, shape, design, configuration, and composition of the CTS may be selected such that, when the CTS is deployed with respect to the tissue site, the CTS, conforms to one or more surfaces of the tissue site, exhibits maximum contact with the tissue site, exhibits minimum contact with the tissue site, exhibits mechanical activity with respect to at least a portion of the tissue site, exhibits activity with respect to a certain microbiome, delivers an active ingredient to the tissue site, meets design criteria specified by the user, or combinations thereof.

In some embodiments, one or more parameters of the CTS may be selected such that the CTS may be characterized as being biodegradable or as exhibiting biodegradability. As used herein, “biodegradable” and “biodegradability” may refer to a characteristic of a material to at least partially break down upon exposure to physiological fluids or processes. For example, in some embodiments, the CTS may disintegrate, degrade, or dissolve when contacted with an aqueous medium, such as water, blood, or wound exudate from a tissue site. Biodegradability may be a result of a chemical process or condition, a physical process or condition, or combinations thereof. Additionally or alternatively, in some embodiments, the CTS may be characterized as being bioresorbable or as exhibiting bioresorbability. As used herein, “bioresorbable” and “bioresorbability” may refer to a characteristic of a material to be broken down into degradation products that may be assimilated at a tissue site so as to be eliminated by the body, for example via metabolism or excretion. In some embodiments, the bioresorbable characteristics of the CTS may be such that at least a portion of the CTS or the material from which the CTS is formed may be eliminated by bioresorption from the tissue site to which it is applied.

Not intending to be bound by theory, the rate, duration, and/or extent of degradation of the CTS may be at least partially dependent upon the composition of the CTS, the sizing (e.g., volume thickness) of the CTS, environmental factors at the site of deployment, or combinations thereof. In some embodiments the dimensions/size, shape, design, configuration, and composition of the CTS may be further selected such that, when the CTS is deployed with respect to the tissue site, the CTS exhibits a desired biodegradation profile and/or exhibits biodegradation within a desired timeframe.

For example, in some embodiments, one or more parameters of the CTS may be selected such that the CTS will exhibit a particular proportion of disintegration, degradation, or dissolution within a particular time period. For instance, in various embodiments the CTS may be configured such that at least 50%, or at least 60%, or at least 70%, or at least 80%, or at least 90%, or at least 95%, or at least 97.5%, or at least 98%, or at least 99%, or at least 99.5%, or at least 99.9% of the volume forming the CTS may degrade and/or biodegrade within a suitable duration of time. For example, in some embodiments, the materials utilized to form the CTS may degrade and/or biodegrade to a such a desired extent in a duration ranging from (A) not less than about 36 hours, or not less than about 72 hours, or not less than about 96 hours, or not less than about 5 days, or not less than about 7 days, or not less than about 14 days, or not less than about 21 days, or not less than about 28 days, or not less than about 1 month, or not less than about 2 months, or not less than about 3 months, or not less than about 4 months, or not less than about 6 months, or not less than about 8 months, or not less than about 10 months, or not less than about 12 months to (B) not more than about 7 days, or not more than about 14 days, or not more than about 21 days, or not more than about 28 days, or not more than about 1 month, or not more than about 2 months, or not more than about 3 months, or not more than about 4 months, or not more than about 6 months, or not more than about 8 months, or not more than about 10 months, or not more than about 12 months, or not more than about 15 months, or not more than about 18 months, or not more than about 24 months. Reference herein to a particular rate and/or duration of degradation associated with the CTS should not be construed as indicating that the CTS must be placed (e.g., in vivo) in order to ascertain such rate and/or duration. For example, a rate and/or duration of degradation may obtained utilizing a suitable test protocol designed to mimic in vivo conditions at and/or proximate to the intended site of deployment and, for example, taking into consideration factors including, but not necessarily limited to, the intended site of deployment, ambient conditions at the intended site of deployment (e.g., temperature, moisture, pH, physical interaction with tissue(s), air-flow), or combinations thereof.

In some embodiments, one or more parameters of the CTS may be selected such that the CTS may be configured to degrade at a desired rate. For example, in some embodiments, one or more parameters of the CTS may be selected such that the CTS may be configured to degrade at a substantially constant rate. Alternatively, in some embodiments, one or more parameters of the CTS may be selected such that the CTS may be configured to degrade at a rate that varies over time. For example, in some embodiments, one or more parameters of the CTS may be selected such that the CTS may degrade at a generally increasing rate; alternatively, in some embodiments the CTS may degrade at a generally decreasing rate. For example, in some embodiments, the CTS may comprise a single degradable material that is degraded at a rate that varies over time. In some embodiments, the CTS comprises multiple degradable materials, for example, having differing rates of degradation, such that the CTS may be degraded at a rate that varies. For example, one or more degradable materials may form in a plurality of layers, together forming the CTS (e.g., a first layer of a first degradable material and a second layer of a second degradable material).

Additionally, in some embodiments, one or more parameters of the CTS may be selected such that the rate of degradation may be altered via the addition or subtraction of a degradation-rate modifying agent. For example, in some embodiments, one or more parameters of the CTS may be selected such that the CTS may exhibit a first (e.g., relatively slow) rate of degradation prior to exposure to and/or contact with a degradation accelerator and to exhibit a second (e.g., relatively fast) rate of degradation upon exposure to and/or contact with such a degradation accelerator. In such an embodiment, the degradation accelerator may comprise an enzymatically-active solution, an irrigating solution (e.g., a hypotonic) saline solution, ultra-violet (UV) radiation, sonic pulsation, or combinations thereof. For example, addition of and/or exposure to such a degradation accelerator may have the effect of causing the CTS to rapidly degrade or “self-destruct.” Alternatively, in some embodiments, one or more parameters of the CTS may be selected such that the CTS will exhibit a first (e.g., relatively slow) rate of degradation while exposed to and/or in contact (e.g., either constantly or intermittently) with a degradation retarder and will exhibit a second (e.g., relatively fast) rate of degradation upon ceasing to be exposed to and/or in contact with such a degradation retarder. For example, removal of and/or ceased exposure to such a degradation retarder may have the effect of causing the CTS to rapidly degrade or self-destruct. Additionally, in some embodiments, one or more parameters of the CTS may be selected such that the CTS will include a degradation-rate modifying agent (e.g., accelerator and/or retarder) may be included within the CTS. For example, the degradation-rate modifying agent may be encapsulated, microencapsulated, or the like, for example, such that the capsules/microcapsules may burst, rupture, or otherwise be caused to release the degradation-rate modifying agent upon a suitable stimulus.

One or more parameters of the CTS may be selected such that the CTS may degrade by any suitable mode of action. Without limitation the mechanisms of polymer degradation may involve hydrolysis, oxidation, aminolysis, enzymatic degradation (e.g., proteolytic degradation), physical degradation, surface erosion (e.g., characterized by a layer by layer degradation of the CTS) bulk erosion, or combinations thereof. Modes of degradation may be affected through the use of external stimuli such as temperature, light, or heat. Additionally or alternatively, degradation of the CTS may occur through contact with one or more materials that facilitate chemical degradation of the CTS.

Referring again to the embodiment of FIG. 1, in some embodiments the CTS modeling system 130 may receive the 3-D data with respect to the tissue site, for example, images from the medical imaging device 120. For example, in some embodiments the CTS modeling system 130 may receive a plurality of image segments. In such embodiments, the CTS modeling system 130 may be configured to utilize the image segments to generate a 3-D model of the tissue. Additionally or alternatively, the 3-D data with respect to the tissue site may undergo various processing prior to being communicated to the CTS modeling system 130. For example, in some embodiments the CTS modeling system 130 may receive a 3-D model of the tissue.

Additionally or alternatively, in some embodiments the CTS modeling system 130 may receive environmental data with respect to the tissue site from the microbiome diagnostic equipment 110. For example, in some embodiments the CTS modeling system 130 may receive data indicating the microbiome, for example, a pathogenic or infectious agent, at, associated with, or proximate the tissue site; the identity or classification of microbiome (e.g., a pathogen) present at or proximate to the tissue site, for example, a viral pathogen, a bacterial pathogen, a fungal pathogen, a prionic pathogen, a protozoal pathogen, a parasitic pathogen, or another potentially-disease-causing micro-organism; the pathogenicity (e.g., amount and/or activity) of any pathogens present at or near the tissue site; the pH of the tissue site; the presence of contaminants at the tissue site; the health of the tissue site; or combinations thereof.

Additionally or alternatively, in some embodiments the CTS modeling system 130 may receive data from a user, such as a physician. For example, the user may provide various inputs to the CTS modeling system such as identification of tissue site at which the CTS is intended to be deployed, one or more limitations with respect to the design, the shape, the configuration, or the composition of the CTS or the intended interaction between the CTS and the tissue site, or combinations thereof.

In some embodiments, the CTS modeling system 130 may be configured to select, for example, via the implementation of one or more selection algorithms or protocols, one or more of the parameters for the CTS on the basis of the data received by the CTS, for example, the 3-D data, the environmental data, and the user inputs. More particularly, the CTS modeling system may consider, not only certain particular data that has been provided, but the totality of the 3-D data, the environmental data, and the user input in the determination of the parameters for the CTS.

In some embodiments, the CTS modeling system 130 may be configured to base one or more parameters for the CTS upon a template, for example, which may be associated with the tissue site where the CTS is intended to be deployed. In some embodiments, a user, such as physician, may select a template upon which the model may be based, for example, from among two or more available templates or designs. In some embodiments, the templates may be based upon various designs for a given configuration of a stent, splint, implant, or other therapeutic support.

For example, in some embodiments, a template may be based upon a standard or conventional design for a given configuration of a stent, splint, implant, or other therapeutic support, for example, which may be specific to various types of tissues sites and/or various implementations. As an example, in the context of a CTS intended for deployment within the nasal cavity, the model may be based upon a conventional Doyle-type nasal splint (e.g., a hollow, cylindrical (e.g., a tube-like) component and a flap extending substantially tangentially from the hollow, cylindrical component) or a conventional a Reuter bivalve-type nasal splint (e.g., a generally planer body having a lengthwise slit). In embodiments where one or more parameters are based upon a template, various characteristics or parameters of the template may be modified to generate the model of the CTS. Alternatively, in some embodiments the parameters may be selected on the basis of the 3-D data, the environmental data, and/or any user inputs, for example, without having any particular feature or parameter based upon any prior design or template.

In some embodiments, a parameter that is ultimately selected for the CTS may deviate from an optimum value. For example, in some embodiments, the selection of certain parameters of the CTS may influence and/or limit certain other parameters thereof. For example, in some embodiments the composition, dimensions, shape, design, configuration, strength characteristics, biodegradation characteristics of the CTS, among others, may be interrelated. In some embodiments, an optimum value or feature for a first parameter may be offsetting with respect to an optimum value or feature for a second parameter. As an example, it may be desirable both to maximize the amount of material used in forming the CTS for purposes of improving strength and stability and, at the same, to minimize the amount of material for purposes of achieving a desired biodegradation time period. In such embodiments, the CTS modeling system 130 may be configured to select a parameter taking into account potentially offsetting features, for example, weighing the importance of various features against each other.

In some embodiments, selecting the one or more parameters for the CTS may include generating a model of the CTS that includes the selected parameters. In various embodiments, the model may stipulate the physical and/or compositional characteristics of the CTS, for example, the size, shape, design, and composition, of the CTS and/or the components thereof. In various embodiments, the model may comprise a virtual representation of the CTS, for example, a 3-D representation of the CTS.

For example, in some embodiments, the model may comprise a stereolithography (STL) file, a CAD file, or an additive manufacturing file (AMF). The CAD file may convey various parameters for the CTS, for example, size, shape, design, and composition, of the CTS and/or the components thereof. In some embodiments, the CAD file may be suitable for displaying and viewing a representation of the CTS and/or fabricating the CTS.

In some embodiments, the method may include fabricating the CTS that includes the selected parameters. For example, in the embodiment of FIG. 3, the method 300 includes fabricating the CTS to have the selected parameters 340.

In some embodiments, fabricating the CTS includes additively manufacturing the CTS, such as by 3-D printing. Generally, in such additive manufacturing processes, layers of a material are selectively joined or fused together in a predetermined patterns to yield particular 3-D objects, particularly, the CTS. The material forming the various layers may be selected such that, when joined or fused, the resultant object (e.g., the CTS) conforms to the selected parameters. In various embodiments, examples of a material or materials that may be used in the additive manufacturing process may include polymers, powders such as a resins and plasters, metals and/or alloys, and combinations thereof. The particular material or materials utilized will vary according to the particular parameters selected with respect to the CTS being fabricated.

Additionally or alternatively, in some embodiments fabricating the CTS includes subtractively manufacturing the CTS, such as by CNC milling (e.g., CNC routing) and laser cutting. Generally, in such subtractive manufacturing processes, predetermined portions of material are removed from a blank to yield particular 3-D objects, particularly, the CTS. The blank used in such subtractive manufacturing may be selected such that, when machined, the resultant object (e.g., the CTS) conforms to the selected parameters.

Additionally or alternatively, in some embodiments an additive or subtractive manufacturing processes, as described herein, may be used to fabricate a mold, for example, which may be used to form the CTS. For example, a material may be injected into a custom mold to form the CTS.

In various embodiments, a CTS fabricated according to the disclosed methods generally comprises a structure having a shape, design, and configuration according to the selected parameters. Examples of shapes, designs, and configurations that may be suitable, according to the particular needs, are disclosed herein.

As an example, in some embodiments the CTS may be formed from one or more strands. For example, the CTS may comprise a plurality of strands may be interconnected or interrelated strands forming relatively simple or relatively complex geometrical structures. In such embodiments, the strands may take the form of filaments, ribbons, tapes, bands, threads, or the like. In such embodiments, the strands may comprise a suitable cross-sectional shape. For example, the strands may have a substantially flat, round, oval, square, triangular, rectangular, tear-drop-shaped, diamond, hollow, or other suitable shape or cross-section, or combinations thereof. In some embodiments, a strand or strands may have a suitable size, for example, with respect to thickness and/or length. For example, in some embodiments, the strands may have a thickness in the range of from about 0.5 mm to about 5 mm, or from about 1 mm to about 4 mm, or from about 2 mm to about 3 mm. Also, in some embodiments the strands may have a length that ranges dependent upon the intended use of the CTS, the configuration of the CTS, and various other parameters.

For example, in some embodiments a plurality of strands may collectively form the CTS or a portion thereof. For example, the CTS may include one or more strands having a generally undulating (e.g., sinusoidal in appearance) configuration, for example having a plurality of “peaks” and “troughs.” These “peaks” and “troughs” may refer to the alternating points of inflection of the undulating strands, but do not necessarily denote any particular absolute orientation (e.g., up or down) of such strands. The one or more strands may be positioned such that the long axis of each of the strands are substantially parallel and offset such that the peak of a given strand may be joined to the trough of an adjacent strand.

Additionally or alternatively, in some embodiments, the one or more strands comprise a strut in the form of a helical segment (e.g., a portion of a helix). For example, a particular strand (e.g., strut) may be joined to another strand (e.g., an adjacent strut) at the terminal ends thereof at a suitable angle, α. Such a suitable angle, α, may be in the range of from about 10 degrees to about 140 degrees.

In another embodiment, the CTS may comprise a single strand. For example, the CTS may be formed from a strand (e.g., a continuous strand) in the shape of a helix. In some embodiments, such a helical strand may exhibit any suitable pitch and/or helical angle. Additionally, such a helical strand may exhibit a constant pitch and/or helical angle; alternatively, such a helical strand may exhibit a variable pitch and/or helical angle.

In some embodiments one or more strands may be woven and/or interlaced together to form the CTS. In such embodiments, the plurality of strands may be woven and/or interlaced at any suitable spacing, in any suitable pattern, and/or at any suitable angle.

Additionally or alternatively, in some embodiments the CTS may be formed from a sheet, a film, a membrane, or the like. For example, in such embodiments, the sheet, film, or membrane may generally comprise a solid material. In some embodiments, the sheet, film, or membrane may be porous. In some embodiments, the sheet, film, or membrane may be characterized as having a suitable size, for example, thickness, width, and/or length. For example, in some embodiments, the sheet may have a thickness in the range of from about 0.5 mm to about 5 mm, or from about 1 mm to about 4 mm, or from about 2 mm to about 4 mm. In various embodiments, the sheet, film, or membrane may have a width and/or length that ranges dependent upon the intended use of the CTS. In some embodiments, the CTS may include the sheet, film, or membrane in a single layer; alternatively, in some embodiments, the CTS may include the sheet, film, or membrane in multiple layers.

In some embodiments, the CTS may comprise any suitable form and/or configuration, for example, as may be dependent upon the intended use of the CTS. In some embodiments, the CTS may be characterized as exhibiting a desired degree of flexibility, conformability, malleability, or rigidity. In some embodiments, the CTS may be characterized as mechanically or structurally active. For example, in some embodiments, at least a portion of a CTS may be configured such that the CTS will apply a radially outward force (e.g., a biasing force), such as in some embodiments where the CTS is deployed within a lumen, as will be disclosed herein. Alternatively, in some embodiments, at least a portion of a CTS may be configured such that the CTS will apply a radially inward force (e.g., a biasing force), such as in some embodiments where the CTS is deployed around (e.g., at least partially encompassing) a tissue, as will be disclosed herein.

In some embodiments, the CTS may be radially expandable and/or contractible with respect to a longitudinal axis or a central passage. For example, in some embodiments, the CTS may be configured so as to expand radially outward and/or to exhibit a radially outward force or bias (e.g., a radially outward mechanical activity) with respect to the longitudinal axis when uninhibited from such expansion, as will be disclosed herein. Alternatively, in some embodiments the CTS may be configured to contract radially inward and/or the exhibit a radially inward force or bias (e.g., a radially inward mechanical activity) with respect to the longitudinal axis when uninhibited from such contraction, as will also be disclosed herein.

In some embodiments the CTS comprises a hollow, generally cylindrical structure having a longitudinal axis. In some embodiments, a hollow, cylindrical CTS may comprise a relatively constant diameter with respect to the longitudinal axis. Alternatively, in some embodiments a hollow, generally cylindrical CTS may comprise a diameter that varies over its length. For example, a hollow, cylindrical CTS may be tapered at one or both longitudinal ends, flared at one or both ends, at least partially conical, or combinations thereof.

In some embodiments, where the CTS comprises a hollow, cylindrical CTS, the CTS may be characterized exhibiting mechanical activity. For example, in some embodiments the CTS may be radially expandable, for example, the CTS may be configured so as to expand radially outward when not retained in a relatively unexpanded conformation. In such embodiments, the CTS may be configured to apply a radially-outward force (for example, to a lumen, passageway, or other opening) upon deployment. In some embodiments, the radially outward force applied by the CTS may be varied according to the particular, selected parameters for the CTS. Additionally, in some embodiments the radially outward force applied by the CTS may be variable over time, according to the particular, selected parameters for the CTS. For example, in some embodiments, the radially outward force applied by the CTS may increase, alternatively, decrease, over time.

In some embodiments, the CTS, or portions or components thereof may comprise a “locking” profile,” such as teeth, grooves, or the like, configured to lock in an expanded or contracted state upon deployment. For example, in some embodiments the CTS may be radially contractible, for example, the CTS may be configured so as to expand radially inward when not retained in a relatively expanded conformation. In such embodiments, the CTS may be configured to apply a radially-inward force (for example, to occlude or compress or narrow a lumen, passageway, or other opening) upon deployment, as will be disclosed herein. In some embodiments, the radially inward force applied by the CTS may be varied, for example, dependent upon the intended use of the CTS, the site of deployment, or other factors as may be appreciated by one of skill in the art upon viewing this disclosure. Additionally, in some embodiments the radially inward force applied by the CTS may be variable over time, for example, as may result from the degradation of a portion of the CTS. For example, in some embodiments, the radially inward force applied by the CTS may increase, alternatively, decrease, over time.

In various embodiments, a CTS fabricated according to the disclosed methods generally comprises composition according to the selected parameters. Examples of compositions that may be suitable, according to the particular needs, are disclosed herein.

In some embodiments, the CTS may comprise a suitable material or composition, for example, a biodegradable composition. For example, in some embodiments, the components and/or materials utilized to form the CTS may comprise one or more polymers, particularly, one or more biodegradable (e.g., bioresorbable) polymers. In various embodiments, the selection of the polymer and/or the combination of polymers utilized may be dependent, at least in part, upon the intended use of the CTS; the desired rate, duration, and/or extent of biodegradation; or combinations thereof.

In some embodiments, the polymer may be present within the CTS in a range of at least about 5%, at least about 10%, at least about 20%, at least about 30%, at least about 40%, at least about 50%, at least about 60%, at least about 70%, at least about 80%, at least about 90%, at least about 95%, or at least about 100% by total weight of such components and/or materials.

Examples of a polymer (e.g., a degradable polymer) that may be suitable for use in making the CTS may include, but are not limited to, a poly(lactide); a poly(glycolide); a poly(lactide-co-glycolide); a poly(lactic acid); a poly(glycolic acid); a poly(lactic acid-co-glycolic acid); poly(lactide)/poly(ethylene glycol) copolymers; a poly(glycolide)/poly(ethylene glycol) copolymer; a polyhydroxy-alkanoate, a poly(lactide-co-glycolide)/poly(ethylene glycol) copolymer; a poly(lactic acid)/poly(ethylene glycol) copolymer; a poly(glycolic acid)/poly(ethylene glycol) copolymer; a poly(lactic acid-co-glycolic acid)/poly(ethylene glycol) copolymer; a poly(caprolactone); poly(caprolactone)/poly(ethylene glycol) copolymer; a poly(orthoester); a poly(phosphazene); a poly(hydroxybutyrate) or a copolymer including a poly(hydroxybutyrate); a poly(lactide-co-caprolactone); a polycarbonate; a polyesteramide; a polyanhidride; a poly(dioxanone); a poly(alkylene alkylate); a copolymer of polyethylene glycol and a polyorthoester; a biodegradable polyurethane; a poly(amino acid); a polyetherester; a polyacetal; a polycyanoacrylate; a poly(oxyethylene)/poly(oxypropylene) copolymer, polysuccinimde, chitin, chitosan, lignosulfonates; chitins; a polysaccharide, a polyphosphazene, a protein, a lipid, or combinations thereof. In some embodiments, such a combination may take the form of a co-polymer and/or a physical blend.

In some embodiments, a polymer suitable for use in the present disclosure is a natural polymer such as protein-based polymers and polysaccharides. Examples of such polymers include collagen, albumin, gelatin, agarose, alginate, carrageenan, hyaluronic acid, dextran, chitin, chitosan, and cyclodextrins. In some embodiments the polymer is a polyanhydride such as poly(sebacicid), poly(adipic acid), and poly(terphtalic) acid. In some embodiments, the polymer is a polyamide such as a poly(imino carbonate) or a polyaminio acid. In some embodiments, the polymer is phosphorus based such as a polyphosphate, a polyphosphonate or a polyphosphazene. In some embodiments, the polymer comprises an α-prolamin.

In some embodiments where the polymer comprises chitosan, the CTS may comprise at least about 40%, or at least about 50%, or at least about 60%, or at least about 70%, or at least about 80%, or at least about 85%, or at least about 90%, or at least about 95% chitosan, by weight of the CTS. The chitosan may be from about 5% to about 50% acetylated, or from about 10% to about 30% acetylated, or from about 12 to about 18% acetylated.

In some embodiments, the polymer comprises a polysaccharide, examples of which may include starches, cellulose, dextran, substituted or unsubstituted galactomannans, guar gums, high-molecular weight polysaccharides composed of mannose and galactose sugars, heteropolysaccharides obtained by the fermentation of starch-derived sugar (e.g., xanthan gum), diutan, scleroglucan, derivatives thereof, or combinations thereof.

In some embodiments, the polymer comprises guar or a guar derivative. Examples of guar derivatives suitable for use in the present disclosure include without limitation hydroxypropyl guar, carboxymethylhydroxypropyl guar, carboxymethyl guar, hydrophobically modified guars, guar-containing compounds, or combinations thereof.

In some embodiments, the polymer comprises cellulose or a cellulose derivative. Examples of cellulose derivatives suitable for use in the present disclosure include without limitation cellulose ethers, ethyl cellulose, cellulose acetate, cellulose acetate propionate carboxycelluloses, carboxyalkylhydroxyethyl celluloses, hydroxyethylcellulose, hydroxypropylcellulose, carboxymethylhydroxyethylcellulose, carboxymethylcellulose, or combinations thereof.

In some embodiments, the polymer comprises a starch. Examples of starches suitable for use in the present disclosure include without limitation native starches, reclaimed starches, waxy starches, modified starches, pre-gelatinized starches, and combinations thereof.

In some embodiments, the polymer comprises a biodegradable shape memory polymer, such as those commercialized by nmemoScience in Aachen, Germany, or those described in U.S. Pat. Nos. 5,189,110 or 5,139,832, each of which is disclosed herein in its entirety.

In some embodiments, the polymer may be made by any suitable process and/or combination of processes. For example, in some embodiments a degradable polymer may be made or otherwise formed in a process comprising melt polymerization. For example, in some embodiments where the polymer comprises one or more monomers having a “ring” structure (e.g., a poly(lactide), a poly(glycolide), a poly(lactide-co-glycolide), a poly(lactic acid), a poly(glycolic acid), and/or a poly(lactid acid-co-glycolic acid)), melt polymerization may result in the opening of such ringed structure. In some embodiments, one or more of the degradable polymers, for example, may be available with or, alternatively, without carboxylic acid end groups. In some embodiments where the end group comprises an ester), the resultant polymer may be referred to herein as a blocked or capped polymer. In some embodiments where the degradable polymer has a terminal carboxylic group, the resultant polymer may be referred to as an unblocked or uncapped polymer. In some embodiments, the degradable polymers may be characterized as linear polymers, star polymers, or combinations thereof. Additionally, in some embodiments, the degradable polymers may be characterized as high molecular weight polymers. For example, not intending to be bound by theory, such high molecular weight polymers may improve the strength of the CTS and/or extend the time duration association with degradation and/or biodegradation (e.g., bioabsorption or resorption time). In some embodiments, low molecular weight polymers may be utilized where degradation and/or biodegradation time is preferably less and/or where less strength is necessary. As will be appreciated by those of skill in the art, lactide monomers and/or the lactide portion of a given polymer comprise an asymmetric carbon. Suitable, racemic DL-, L-, and D-polymers, for example, as may be suitable for inclusion in the composition, are commercially available. Not intending to be bound by theory, the L-polymers may be relatively more crystalline and, as such, may degrade and/or biodegrade (e.g., adsorb, absorb, resorb, and/or dissipate) more slowly than DL-polymers. Copolymers comprising glycolide and DL-lactide or L-lactide as well as copolymers of L-lactide and DL-lactide are also commercially available. Additionally, homopolymers of lactide or glycolide are commercially available. Star polymers of lactide or glycolide or lactide/glycolide copolymers are also commercially available.

In some embodiments where the polymer comprises a poly(lactide-co-glycolide), a poly(lactide), or a poly(glycolide), the lactide and/or the glycolide may be present within the polymer in a suitable amount. In some embodiments, the degradable polymer contains a lactide in an amount of from about 0 to about 100 mole %, or from about 40 to about 100 mole %, or from about 50 to about 100 mole %, or from about 60 to about 100 mole %, or from about 70 to about 100 mole %, or from about 80 to about 100 mole %, and/or glycolide in an amount of from about 0 to about 100 mole %, or from about 0 to about 60 mole %, or from about 10 to about 40 mole %, or from about 20 to about 40 mole %, or from about 30 to about 40 mole %. In some embodiments, the degradable polymer contains lactide and glycolide in an amount summing 100 mole %, or about 99%, or about 95%, or about 90%. In some embodiments where the degradable polymer comprises lactide and glycolide, the lactide and glycolide may be present in a ratio of about 85:15 poly(lactide-co-glycolide), or about 75:25 poly(lactide-co-glycolide), or about 65:35 poly(lactide-co-glycolide), or about 50:50 poly(lactide-co-glycolide), where the ratios are mole ratios.

In an additional and/or alternative embodiment, the polymer may comprise a nondegradable polymer. Examples of a suitable nonbiodegradable polymer include, but are not limited to, poly(ethylene vinyl acetate), poly(vinyl acetate), silicone polymers, polyurethanes, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, copolymers thereof, or combinations thereof. For example, in some embodiments the CTS or a portion or component thereof may comprise both degradable and non-degradable portions.

In some embodiments, the CTS may comprise a composition modifier. For example, in some embodiments the CTS may comprise a plasticizer. An example of a plasticizer includes glycerol. When present, the CTS may comprise from about 0.1% to about 10%, by weight of the CTS.

In some embodiments, the polymer (e.g., a degradable or nondegradable polymer) may be characterized as mucoadhesive or bioadhesive. As used herein, the terms mucoadhesive and/or bioadhesive may refer to materials tending to exhibit adhesion to biological tissue, such as mucosae (e.g., one or more mucous membranes). In some instances, the CTS, one or more of the components and/or materials utilized to form the CTS, and/or the CTS may be coated with a mucoadhesive, which may or may not be a polymer. Additionally, in some embodiments the polymer may be characterized as charged and/or ionic in character, for example, may alter the interaction of the polymer(s) with one or more bodily surfaces.

In some embodiments, the composition of the CTS may include an active ingredient (AI). In some embodiments, the AI may be incorporated within the CTS during fabrication (e.g., with and/or within the composition forming the CTS), or within a portion thereof. For example, the AI may be incorporated throughout substantially the entire volume of the CTS or in particular portions thereof. For example, in some embodiments the composition used to form the CTS in an additive manufacturing process (e.g., 3-D printing) may include the AI. Additionally or alternatively, in some embodiments the AI may be disposed within pores or void-spaces within the CTS, such that the AI may be released upon degradation of the CTS. Additionally or alternatively, the AI may be encapsulated and/or microencapsulated and the encapsulated/microencapsulated AI may be incorporated within the CTS.

In some embodiments, the CTS may be configured such that the one or more AIs may be eluted differently with respect to time. For example, in some embodiments, a single AI may be added to the CTS such that the AI is eluted at a rate that varies over time. In some embodiments, multiple AIs may be added to the CTS such that the multiple AIs are eluted at independently variable rates. For example, one or more AIs may be added to the CTS in a plurality of layers, within pockets, in reservoirs, encapsulated in microspheres which may allow the release of the AI at variable rates, or combinations thereof. Additionally, in some embodiments, a portion of the AI may be quick-release (e.g., configured for immediate or substantially-immediate release from the CTS) and another portion of the AI may be extended release (e.g., configured for release, from the same or a difference portion of the CTS, over a relatively long duration of time).

Additionally or alternatively, in some embodiments fabricating the CTS includes modifying the composition of the CTS after the structure of the CTS has been formed, for example, after the structure has been additively or subtractively manufactured. For example, in some embodiments, the AI may be coated onto the surface of structure of the CTS or a portion thereof. For example, the AI may be dissolved or suspended in a solution, which may be applied to one or more surfaces of the CTS, such as by spraying, dipping, bathing, adsorption, absorption, or the like. Alternatively, the AI may be coated onto one or more surfaces of the CTS as a powder and adhered thereto, for example, by heating the CTS and/or softening the CTS with a plasticizer.

In some embodiments, the AI may comprise any suitable pharmaceutically active component, any suitable active pharmaceutical ingredient (API), any suitable therapeutically-active component, any-suitable prophylactically-active component, any suitable cosmetic component, any suitable material that is safe for human use and has biological activity, or combinations thereof. The AI(s) may be adherent to the CTS itself, or incorporated or otherwise surrounded, encompassed, or sequestered within and/or by the CTS. In some embodiments, the AI(s) may be incorporated into the CTS along with co-ingredients to alter, delay, hasten, or otherwise manipulate or control the rate of release and activity at the tissue site.

In some embodiments, the AI may comprise any ingredient suitable for the treatment, prevention, rehabilitation, therapy, alteration, minimization, amelioration, camouflage, or the like of a condition in a subject (e.g., a human “patient”), examples of which include but are not limited to chronic sinusitis, obstructive sleep apnea, postoperative inflammation/scarring/non-healing wounds, stenosis of a tubular structure due to injury or burn, middle ear inflammatory disease, Eustachian tube dysfunction, nasolacrimal duct obstruction, pharyngeal stenosis, spinal stenosis, spinal rootlet compression, esophageal stenosis, incompetence of the lower esophageal sphincter, laryngeal stenosis, tracheal stenosis, reactive airway disease, biliary tract stenosis, pancreatic duct stenosis, ureteral stenosis, urethral stricture, Fallopian tube obstruction, contraception, sexually-transmitted diseases in the male and the female, interruption of the Fallopian tubes or the vas deferens due to prior contraceptive surgical procedures, vascular occlusion or stenosis, malignancy requiring chemotherapy, physical defects requiring skin grafting or microvascular free flap tissue transfer, temporary or permanent change in eye color, and in rejuvenation of the aging face.

Examples of the AI may include, but are not limited to, anticholinergic agents, anti-infective agents (e.g., an antibiotic, such as antibacterial agents, antifungal agents, antiparasitic agents, antiviral agents, antiseptics or combinations thereof), anti-inflammatory agents (such as steroidal and/or nonsteroidal anti-inflammatory agents), anti-scarring or antiproliferative agents, chemotherapeutic/antineoplastic agents, cytokines, decongestants, extracellular signaling/intracellular signaling molecules, healing-promotion agents and vitamins, hemostatic agents, hormones, hyperosmolar agents, immunoglobulins, immunomodulators, immunosuppressive agents, leukotriene modifiers, mast cell stabilizers, mitotic inhibitors, mucolytics, muscle relaxants, narcotic analgesics, non-narcotic analgesics, nucleic acids, other peptides, other proteins including potential allergens for immunotherapy (e.g., pollen), proton-pump inhibitors, sclerosing agents, tyrosine kinase inhibitors, vasoactive agents, and combinations thereof. Additionally, anti-sense nucleic acid oligomers or other direct transactivation and/or transrepression modifiers of mRNA expression, transcription, and protein production may also be used.

In some embodiments, suitable anticholinergic agents may generally include antimuscarinic agents, ganglionic blockers, neuromuscular blockers, and combinations thereof. Examples of anticholinergic agents include, but are not limited to, ipratroprium bromide, dicycloverine, atropine, benztropine, ipratropium, oxitropium, tiotropium, glycopyrrolate, oxy butnin, tolterodine, diphenhydramine, and dimenhydrinate.

In some embodiments, suitable antihistamines may generally include H₁-receptor antagonists, H₂-receptor antagonists, H₃-receptor antagonists, H₄-receptor antagonists, histidine decarboxylase inhibitors, mast cell stabilizers, and combinations thereof. Examples of suitable H₁-receptor antagonists include, but are not limited to, azelastine, diphenhydramine, chlorpheniramine, meclozine, promethazine, loratadine, desloratadine, fexofenadine, cetirizine, levocetirizine, olopatadine, brompheniramine, buclizine, bromodiphenhydramine, carbinoxamine, chlorpromazine, cyclizine, chlorpheniramine, clemastine, cyproheptadine, dexbromheniramine, deschlorpheniramine, dexchlorpheniramine, dimenhydrinate, dimetindene, doxylamine, ebastine, embramine, orphenadrine, phenindamine, pheniramine, phenyltoloxamine, pyrilamine, quetiapine, rupatadine, tripelennamine, and trirolidine. Examples of suitable H₂-receptor antagonists include, but are not limited to, cimetidine, famotidine, lafutidine, nizatidine, ranitidine, and roxatidine. Examples of suitable H₃-receptor antagonists include, but are not limited to, ciproxifan, clobenpropit, conessine, and thioperamide. Examples of suitable H₄-receptor antagonists include, but are not limited to, thioperamide. Examples of suitable histidine decarboxylase inhibitors include tritiqualine and catechin. Examples of suitable mast cell stabilizers include cromoglicate, medocromil, cromolyn sodium, and β2 adrenergic agonists.

Examples of suitable antibacterial agents include, but are not limited to, aminoglycosides, amphenicols, ansamycins, β-lactams, lincosamides, macrolides, nitrofurans, quinolones (e.g., levofloxacin), sulfonamides, sulfones, tetracyclines, vancomycin, and any of their derivatives, and combinations thereof.

In some embodiments, suitable β-lactams include, but are not limited to, carbacephems, carbapenems, cephalosporins, cephamycins, monobactams, oxacephems, penicillins, and any of their derivatives.

In some embodiments, suitable penicillins include, but are not limited to, amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, and ticarcillin. In some embodiments, penicillins may be combined with clavulanic acid. An example of a suitable combination of a penicillin and clavulanic acid is Augmentin™ (amoxicillin and clavulanic acid).

Examples of suitable antifungal agents include, but are not limited to, allylamines, imidazoles, polyenes, thiocarbamates, triazoles, and any suitable derivatives thereof.

Examples of suitable antiparasitic agents include atovaquone, clindamycin, dapsone, iodoquinol, metronidazole, pentamidine, primaquine, pyrimethamine, sulfadiazine, trimethoprim/sulfamethoxazole, trimetrexate, and combinations thereof.

Examples of suitable antiviral agents include, but are not limited to, acyclovir, famciclovir, valacyclovir, edoxudine, ganciclovir, foscamet, cidovir (vistide), vitrasert, formivirsen, HPMPA (9-(3-hydroxy-2-phosphonomethoxypropyl)adenine), PMEA (9-(2-phosphonomethoxyethyl)adenine), HPMPG (9-(3 -Hydroxy-2-(Phosphonomethoxy)propyl)guanine), PMEG (9-[2-(phosphonomethoxy)ethyl](guanine), HPMPC (1-(2-phosphonomethoxy-3-hydroxypropyl)-cytosine), ribavirin, EICAR (5-ethynyl-1-beta-D-ribofuranosylimidazole-4-carboxamine), pyrazofurin (3-[beta-D-ribofuranosyl]-4-hydroxypyrazole-5-carboxamine), 3-Deazaguanine, GR-92938X (1-beta-D-ribofuranosylpyrazole-3,4-dicarboxami-de), LY253963 (1,3,4-thiadiazol-2-yl-cyanamide), RD3-0028 (1,4-dihydro-2,3-Benzodithiin), CL387626 (4,4′-bis[4,6-d]3-aminophenyl-N-,N-bis(2-carbamoylethyl)-sulfonilimino]-1-,3,5-triazin-2-ylamino-biphenyl-2-,2′-disulfonic acid disodium salt), BABIM (Bis[5-Amidino-2-benzimidazoly-1]-methane), NIH351, and combinations thereof.

In some embodiments, an anti-inflammatory agent may comprise a steroidal anti-inflammatory agent (e.g., a corticosteroid). Examples of suitable steroidal anti-inflammatory agents include 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, any of their derivatives, and combinations thereof.

Additionally or alternatively, in some embodiments the anti-inflammatory agent may comprise a nonsteroidal anti-inflammatory agent. Examples of suitable nonsteroidal anti-inflammatory agents include, but are not limited to, COX inhibitors (COX-1 or COX nonspecific inhibitors) (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam) and alkanones such as nabumetone); selective COX-2 inhibitors (e.g., diaryl-substituted furanones such as rofecoxib; diaryl-substituted pyrazoles such as celecoxib; indole acetic acids such as etodolac and sulfonanilides such as nimesulide); and combinations thereof.

Examples of anti-scarring agents include, but are not limited to, silicon and vitamin E and derivatives thereof.

Examples of antiseptics include, but are not limited to chlorhexadine, benzalkonium chloride, octenidine, boric acid, cetyl trimethylammonium bromide, cetylpyridium chloride, benzethonium chloride, triclosan, sucralfate, quaternatry ammonium salts, betadine, polyhexanide, iodine and derivatives thereof, silver, and combinations thereof. When present, the silver may be metallic in form, ionic in form (e.g., a silver salt), or both. Additionally or alternatively, in some embodiments a natural material or substance having desirable antibiotic and/or antiseptic properties (e.g., Manuka Honey) may be included as an antiseptic.

Examples of suitable chemotherapeutic/antineoplastic agents include, but are not limited to antitumor agents (e.g., cancer chemotherapeutic agents, biological response modifiers, vascularization inhibitors, hormone receptor blockers, cryotherapeutic agents or other agents that destroy or inhibit neoplasia or tumorigenesis) such as alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6 mercaptopurine and 5-fluorouracil (5FU), antitumor antibiotics and other compounds that act by binding or intercalating DNA and preventing RNA synthesis (e.g., doxorubicin, daunorubicin, epirubicin, idarubicin, mitomycin-C and bleomycin) plant (vinca) alkaloids and other anti-tumor agents derived from plants (e.g., vincristine and vinblastine), steroid hormones, hormone inhibitors, hormone receptor antagonists and other agents which affect the growth of hormone-responsive cancers (e.g., tamoxifen, herceptin, aromatase ingibitors such as aminoglutethamide and formestane, trriazole inhibitors such as letrozole and anastrazole, steroidal inhibitors such as exemestane), antiangiogenic proteins, small molecules, gene therapies and/or other agents that inhibit angiogenesis or vascularization of tumors (e.g., meth-1, meth-2, thalidomide), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAI), combretastatin A4 prodrug (CA4P), SU6668, SU11248, BMS-275291, COL-3, EMD 121974, IMC-1C11, IM862, TNP-470, celecoxib (Celebrex), rofecoxib (Vioxx), interferon alpha, interleukin-12 (IL-12) or any of the compounds identified in Science Vol. 289, Pages 1197-1201 (Aug. 17, 2000), which is incorporated herein by reference in its entirety, biological response modifiers (e.g., interferon, bacillus calmette-guerin (BCG), monoclonal antibodies, interluken 2, granulocyte colony stimulating factor (GCSF), etc.), PGDF receptor antagonists, herceptin, asparaginase, busulphan, carboplatin, cisplatin, carmustine, cchlorambucil, cytarabine, dacarbazine, etoposide, flucarbazine, flurouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxol or paclitaxel, taxotere, analogs/congeners, derivatives of such compounds, and combinations thereof.

Examples of cytokines include, but are not limited to interferon (such as, but not limited to type I, type II, and type III interferons) and interleukins.

Examples of proton-pump inhibitors include, but are not limited, dexlansoprazole, esomeprazole, lansoprazole, omeprazole, pantoprazole, rabeprazole, and combinations thereof.

Examples of decongestants include, but are not limited to, epinephrine, pseudoephedrine, oxymetazoline, phenylephrine, tetrahydrozolidine, xylometazoline, and combinations thereof.

Examples of healing-promotion agents and vitamins include, but are not limited to, retinoic acid, vitamin A, vitamin D, vitamin E, vitamin K, and derivatives thereof.

Examples of hemostatic agents include, but are not limited to, aminocaproic acid and desmopressin.

Examples of potential hormones include, but are not limited to, androgens (such as aldosterone, testosterone, or dehydroepiandrosterone), anti-androgens, estrogens, anti-estrogens, progesterone, estradiol, GnRH analogs, steroids, sterols, growth hormones, anti-diuretic hormone, melatonin, serotonin, thyroxine, triiodothyronine, calcitonin, thyroid-stimulating hormone (TSH), parathyroid hormone, glucagon, epinephrine, norepinephrine, dopamine, oxytocin, insulin, insulin-like growth factor, and analogs thereof.

In some embodiments where it is desirable to remove water from a tissue (e.g., to remove fluid from polyps or edematous tissue) a hyperosmolar agent may be employed. Examples of suitable hyperosmolar agents include, but are not limited to, furosemide, sodium chloride gel, or other salt preparations that draw water from tissue or substances that directly or indirectly change the osmolar content of the mucous layer.

In some embodiments, immunomodulators may generally comprise immunosuppressants, immunostimulants, tolerogens, or combinations thereof. Examples of immunomodulators include imiquimod, cyclosporine, tacrolimus, azathioprine, cyclophosphamide, methotrexate, chlorambucil, mycophenolate mofetil (MMF), prednisolone, levamisole, and thalidomide.

Examples of leukotriene modifiers include, but are not limited to, montelukast.

Examples of mitotic inhibitors include, but are not limited to, mitomycin-C, taxanes, topoisomerase inhibitors, and vinca alkaloids.

Examples of muscle relaxants include cyclobenzaprine, dantrolene, metaxalone, and tizanidine.

Examples of mucolytics include, but are not limited to, acetylcysteine, domase alpha, guafenesin, and combinations thereof.

Examples of narcotic analgesics include, but are not limited to, codeine, fentanyl, hydrocodone, hydromorphone, meperidine, methadone, morphine, oxycodone, oxymorphone.

Examples of non-narcotic analgesics include, but are not limited to, acetaminophen, aspirin, celecoxib, diclofenac, ibuprofen, indomethacin, ketorolac, misoprostol, meloxicam, naproxen sodium, and combinations thereof.

Examples of vasoactive agents include, but are not limited to, nitrates, vasoconstrictors, vasodilators, and combinations thereof.

In some embodiments, the AI may be present in a suitable form. For example, in some embodiments, the AI may comprise a pharmaceutically acceptable salt, a suitable prodrug, a suitable solvate (e.g., a hydrous crystalline form an anhydrous crystalline form), or derivatives thereof, or combinations thereof.

As used herein, the term “pharmaceutically acceptable salt” refers to salts of active compounds which are prepared with relatively non-toxic acids. Acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic; propionic; isobutyric; maleic; malonic; benzoic; succinic; suberic; fumaric; mandelic; phthalic; benzenesulfonic; toluenesulfonic, including p-toluenesulfonic, m-toluenesulfonic, and o-toluenesulfonic; citric; tartaric; methanesulfonic; and the like. Also included are salts of amino acids such as arginate and the like, and salts of organic acids like glucuronic or galactunoric acids and the like (see, for example, Berge et al. J. Pharm. Sci. 66:1-19 (1977, which is incorporated herein by reference in its entirety)).

As used herein, the term “prodrug,” refers to a compound that is a drug precursor which, upon administration to a subject, undergoes chemical conversion by metabolic or chemical processes to yield a desired compound (e.g., an API) or a salt and/or solvate thereof (e.g., a prodrug on being brought to the physiological pH or through enzyme action is converted to the desired drug form). A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated in their entirety herein by reference.

As used herein, the term “solvate” refers to a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is water.

Generally, the AI may be present in the CTS in a “safe and effective” amount. Safe and effective amounts are sufficient to impart a desired effect (e.g., antimicrobial activity), without undue adverse side effects (such as toxicity, irritation, or allergic response), commensurate with a reasonable benefit/risk ratio when used in the manner of this technology. The specific safe and effective amount of an AI may vary with the particular AI and other factors such as the physical form of the AI, the type and quantity of other materials in the composition, the intended use, and the physical condition of the patient on whom the substrate is intended for use. In general, the AIs may be present at a level of from about 0.1% to about 10%.

In some embodiments, the AI may be eluted (e.g., released) from the CTS at a suitable rate. For example, the CTS may be configured to elute the AI at a rate, per day, in the range of from a first end point (A) to a second end point (B). For example, the CTS may be configured to elute the AI at a rate, per day, in the range of from (A) at least about 0.01 μg, or at least about 0.1 μg, or at least about 1 μg, or at least about 5 μg, or at least about 10 μg, or at least about 25 μg, alternatively at least about 50 μg, or at least about 75 μg, or at least about 100 μg, or at least about 150 μg, or at least about 200 μg, to (B) at most about 10 μg, or at most about 25 μg, or at most about 30 μg or at most about 40 μg, or at most about 50 μg, or at most about 75 μg, or at most about 100 μg, or at most about 200 μg, or at most about 300 μg, or at most to about 400 μg, or at most to about 500 μg, or at most to about 600 μg, or at most to about 700 μg, or at most to about 800 μg, or at most to about 900 μg, or at most to about 1,000 μg, or at most to about 1,250 μg, or at most to about 1,500 μg, or at most to about 2,000 μg, or at any other suitable rate. Not intending to be bound by theory, the rate of elution of the AI may depend upon one or more factors, examples of which include, but are not limited to those factors set forth above.

In some embodiments, the AI may be eluted from the CTS over a suitable period of time, for example, as may be at least partially dependent upon the intended use of the CTS and/or the CTS. For example, in some embodiments the AI may be eluted from the CTS such that at least 50%, or at least 60%, or at least 70%, or at least 80%, or at least 90%, or at least 95%, or at least 97.5%, or at least 98%, or at least 99%, or at least 99.5%, or at least 99.9% of the AI associated with the CTS will be eluted therefrom within a suitable duration of time. For example, in some embodiments, the AI may be eluted to such a desired extent in a duration ranging from (A) a start point of not less than about 72 hours, or not less than about 96 hours, or not less than about 5 days, alternatively not less than about 7 days, or not less than about 14 days, or not less than about 21 days, or not less than about 28 days, or not less than about 1 month, or not less than about 2 months, or not less than about 3 months, or not less than about 4 months, or not less than about 6 months, or not less than about 8 months, or not less than about 10 months, or not less than about 12 months to (B) an end point of about not more than about 7 days, or not more than about 14 days, or not more than about 21 days, or not more than about 28 days, or not more than about 1 month, or not more than about 2 months, or not more than about 3 months, or not more than about 4 months, or not more than about 6 months, or not more than about 8 months, or not more than about 10 months, or not more than about 12 months, or not more than about 15 months, or not more than about 18 months, or not more than about 24 months.

In some embodiments, the duration for AI to be eluted may be less than, alternatively, substantially the same as, alternatively, the same as the duration over which the CTS degrades and/or biodegrades. In some embodiments, the elution of the AI may be contemporaneous with the degradation and/or biodegradation of the CTS. In an alternative embodiment, the elution of the AI may be partially contemporaneous with the degradation and/or biodegradation of the CTS. For example, the elution of the AI may begin substantially contemporaneous with, alternatively, after, biodegradation of the CTS has begun and/or, the elution of the AI may end substantially contemporaneous with, alternatively, before, biodegradation of the CTS has ended.

In some embodiments, a CTS of the type provided according to one or more of the methods disclosed here may be used in the provision of treatment to a patient. “Treatment” refers to an intervention performed with the intention of preventing the development or altering the pathology of such an undesirable condition. Accordingly “treating” may include both to therapeutic treatments and to prophylactic measures.

In some embodiments, a CTS is used in treating a patient in need thereof. In some embodiments, the patient may be generally characterized as experiencing a dysfunction, undesirable medical condition, disorder, or disease state. The dysfunction, undesirable medical condition, disorder, or disease state will be collectively referred to hereinafter as an “undesirable condition.” For example, the undesirable condition may include conditions such as “genetic diseases” which refer to conditions attributable to one or more gene defects. An “undesirable condition” may also include “acquired pathologies” which refer to pathological conditions that are not attributable to inborn defects, cancers, diseases, and the like. Examples of “acquired pathologies” include, but are not limited to, infectious diseases, post-operative states, or traumatic defects. Additionally, an “undesirable conditions” may include structural or functional conditions, for example, that develop throughout life, such as upper airway collapse during inspiration (e.g., as seen in obstructive sleep apnea), chronic Eustachian tube dysfunction, chronic rhinosinusitis, vertebral disk disease, presbyphonia, natural cosmetic aging, peripheral vascular disease, fertility beyond the timeframe of desired reproduction, Type II diabetes mellitus, morbid obesity, or the like. In some embodiments, the undesirable condition may comprise one or more of the conditions or states disclosed herein or any other condition or state as may be appreciated by one skill in the art upon viewing this disclosure.

For example, in some embodiments, the patient may be characterized as having been diagnosed with, experiencing symptoms associated with, wishing to prevent, or otherwise wishing to treat, medicate, correct, lessen the symptoms associated with, a disease, an illness, condition, an abnormality, a predisposition, and/or one or more of the symptoms associated therewith.

Additionally or alternatively, in some embodiments, the patient may be characterized as having recently undergone (or planning to undergo) a corrective procedure, a restorative procedure, an elective procedure, a cosmetic procedure, an urgent procedure, a life-extending procedure, a life-saving procedure, or combinations thereof, any of which may be undertaken or supplemental with a treatment of the type described herein (e.g., use of a CTS in furtherance thereof). For example, in some embodiments a CTS may be employed in ophthalmologic surgery, middle ear surgery, endoscopic sinus surgery, a surgery to improve airway collapse in obstructive sleep apnea, vocal cord surgery, tracheobronchial surgical procedures, gastrointestinal procedures or surgery, urologic surgery, gynecologic surgery for the enhancement or prevention of the likelihood of pregnancy, neurosurgery such as vertebral corrective procedures, endovascular surgery, cosmetic surgery, reconstructive surgery, a surgery to repair traumatic or burn defects, or combinations thereof.

In some embodiments, a method of utilizing or using a CTS may comprise administering the CTS to a patient in need thereof to treat an undesirable condition of the type(s) disclosed herein. In some embodiments, a CTS may be deployed by a physician in a clinical setting. For example, in some embodiments a CTS may be deployed by the physician at, within, about, or otherwise proximate to an intended site of deployment within the patient where an undesirable condition exists. The site of deployment may be the tissue site with respect to with 3-D data and/or environmental data was obtained in selecting the parameters for the CTS.

In some embodiments, the CTS may be compressed and/or contracted (e.g., radially compressed) loaded into a suitable deployment tool (e.g., a plunger or syringe-type deployment tool) configured to selectively retain the CTS in such a radially compressed conformation. The deployment tool may then be brought into the vicinity of the intended site of deployment (e.g., tissue), extruded or otherwise ejected or emptied from the deployment device, and allowed to radially expand. Alternatively, the CTS may be deployed utilizing one or more conventionally-utilized surgical instruments, such as forceps (e.g., bayonet forceps) and/or a speculum (e.g., a nasal speculum). For example, upon providing access to the site of deployment (e.g., with a speculum or other, similar device), the CTS may be brought into the vicinity of the intended site of deployment and released. Upon deployment, the CTS may contact the intended target tissue site, for example, such that the propensity for radial expansion exhibiting by the CTS exerts a force so as to maintain patency of the lumen or other passageway.

In another embodiment, a CTS may be deployed by a physician in conjunction with an operative procedure. For example, using a syringe-like or plunger-like type of deployment tool or any suitable mode of deployment, a surgeon may bring the apparatus, previously having been loaded with a CTS, into the vicinity of the intended target tissue for treatment, and thus deploy the device, for example, during the course of an operative procedure. In some embodiments, a CTS that is conformable, trimmable, and malleable could be brought into the vicinity of a malignant tumor or the vascular inflow of such a malignant tumor, fashioned to conform to this structure, for example, so as both perform occlusive functions and (when an AI is present) targeted AI delivery to the intended treatment site. In another embodiment, a CTS may be deployed post-operatively. For example, in some embodiments, a CTS could be deployed into (e.g., allowed to radially expand within) a lumen or passageway, for example, to maintain postoperative patency for a defined period of time while (when an AI is present) simultaneously delivering AI(s) to aid in therapy and healing of the surgical site.

In still another embodiment, the CTS may be configured to not necessarily expand or contract, but rather provide predictable time-release of AI at an intended target site and/or provide a structural barrier. For example, as will be disclosed herein in greater detail, a CTS may be deployed as an intrauterine device that could release contraceptive hormones, pro-fertility agents such as clomiphene, inhibit implantation of a fertilized egg into the endometrium, or any combination of the above. In such embodiments, as disclosed herein, after the period of time of usefulness, the CTS may be configured to biodegrade via the body's natural degradatory mechanisms.

In some embodiments, a CTS may be deployed within a lumen or other passageway. For example, a CTS may be deployed within a lumen or other passageway for the purpose of providing and/or maintaining patency of such lumen or passageway. In such embodiments, the CTS may be deployed within the external auditory canal, the Eustachian tube, the nasolacrimal system, the upper or lower airway, the esophagus, the gastrointestinal tract, the hepatobiliary tract, the pancreatic duct, the genitourinary tract including the male and female reproductive tracts, intravascularly, inter-vertebrally, within the spinal canal or nerve foramena, at the anastamotic site of a vascular repair such as in a vascular bypass procedure or a microvascular free flap tissue transfer procedure, or in combinations thereof.

In an alternative embodiment, a CTS may be deployed around a tissue or other anatomical structure. For example, a CTS may be deployed around a tissue or other anatomical structure for example, for the purpose of delivering one or more AIs to the anatomical structure, or in the case of a vascular structure, to the anatomical structures downstream from it, for the purpose of occluding partially or completely this tissue, or combinations thereof. For example, in some embodiments, a deployment apparatus could be used by a surgeon to narrow the lower esophagus or the lower ureter in undesirable conditions such as gastroesophageal reflux disease or vesicoureteral reflux disease, respectively. In another embodiment, the CTS could be used for complete, permanent occlusion of an anatomical structure, such as the Fallopian tube, to prevent an egg from reaching the uterus for possible fertilization, in the vas deferens for prevention of sperm reaching the eventual semen and ejaculate, or in the feeding vessel of an aneurysm or varicosity to completely prevent vascular inflow.

Alternatively, in some embodiments, a method of utilizing a CTS may comprise making a CTS available to a patient, for example, for deployment by the patient themselves and/or by another (e.g., a parent or caregiver) in a non-clinical setting. In such embodiments, For example, in some embodiments, a CTS may be configured for deployment by a patient or another. For example, in some embodiments, the patient or another (e.g., a non-physician) may obtain a CTS deployment device. For example, the CTS may be compressed within (e.g., preloaded and/or packaged) such a deployment device such that, the patient may deploy the CTS within a suitable lumen, passageway, or the like. Not intending to be bound by theory, the CTS and/or the deployment device may be configured for safe deployment by the patient themselves, for example, without concern that the CTS will be deployed wrongly, unsafely, or inappropriately.

In an additional embodiment, making the CTS available to a patient may comprise packaging the CTS with instructions, illustrations, or the like, depicting deployment procedures. Also, in some embodiments, making the CTS available to a patient may comprise packaging the CTS with a deployment device (e.g., a preloaded plunger-like or syringe-like device), for example, allowing for deployment by the patient and/or another.

In some embodiments, the CTS may be deployed with respect to the tissue site within a relatively short duration from one or more steps by which the CTS is provided. For example, in some embodiments, the steps of characterizing a tissue site or a portion thereof, selecting parameters for the CTS on the basis of the characterization of the tissue site, fabricating the CTS and, thereafter, deploying the CTS at the tissue site, may all take place within a single visit to a clinic by the patient. For example, the presence of imaging equipment, diagnostic equipment and fabrication equipment at a clinic may enable a physician to provide a CTS even within the course of a procedure.

In some embodiments, the CTS modeling system 130 may be updated based upon the outcome of a previously-deployed CTS. For example, in some embodiments the CTS modeling system 130 may implement one or more algorithms, protocols, or logical tasks in the performance of selecting the parameters of a CTS and the CTS modeling system 130 may update those algorithms, protocols, or logical tasks such that, when implemented, the CTS modeling system 130 may will more nearly achieve the desired result or criteria when determining selecting the parameters for a future CTS. In some embodiments, the CTS modeling system 130 may utilize one or more techniques associated with artificial intelligence in order to improve the CTS modeling system 130 may, for example, machine learning. As used herein, “machine learning” generally refers to any computer-implemented statistical technique by which a computer or computer system is able to progressively improve the performance (e.g., the results associated with) a particular task or goal. Examples of suitable machine-learning methodologies, as may be suitably employed to update the CTS modeling system 130 may (for example, such that when implemented, the CTS modeling system 130 may will more nearly achieve the desired result or criteria when determining selecting the parameters for a future CTS) may include, but are not limited to decision tree learning; rule-based learning; association rule learning; artificial neural networks and deep learning; support vector machine learning; cluster analysis; representation learning; learning classification systems; and the like.

In various embodiments, by utilizing a suitable machine learning technique, CTS modeling system 130 may alter (e.g., optimize) the CTS modeling system 130 may based upon various feedback data associated with a previously-deployed CTS, for example, the degradation period observed, the presence of absence of an infection or other pathogen, the healing observed at the tissue site, whether the CTS achieved an intended result, a follow-up assessment by a physician, an assessment by the patient, or the like. Based upon these updates to the CTS modeling system 130, the CTS modeling system may alter the way in which parameters are selected during the modeling of a CTS.

One of skill in the art, upon viewing this disclosure, will appreciate one or more additional embodiments and/or variations of a CTS as disclosed herein and/or methods of utilizing the same. As such, the forgoing is in no way intended to be limited to the embodiments or example disclosed herein.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rl, and an upper limit, Ru, is disclosed, any number falling within the range is specifically disclosed. In particular, the following numbers within the range are specifically disclosed: R=Rl+k*(Ru−Rl), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present invention.

What is claimed is:

1. A method of providing therapy to a tissue site, the method comprising:
collecting three-dimensional (3-D) data associated with the tissue site;
selecting at least one parameter of a customized tissue support based upon the 3-D data;
fabricating the customized tissue support including the design parameter.

2. The method of claim 1, wherein the tissue site includes at least a portion of the nasal cavity.

3. The method of claim 1, wherein the tissue site includes the paranasal sinuses.

4. The method of claim 1, wherein the 3-D data comprises a physical dimension associated with the tissue site.

5. The method of claim 1, wherein the 3-D data indicates the presence of an abnormality.

6. The method of claim 1, wherein collecting the 3-D data comprises imaging the tissue site.

7. The method of claim 6, wherein imaging the tissue site comprises performing a computed tomography (CT) scan of the tissue site.

8. The method of claim 6, wherein imaging the tissue site comprises performing a magnetic resonance imaging scan of the tissue site.

9. The method of claim 6, wherein imaging the tissue site comprises performing an ultrasound scan of the tissue site.

10. The method of claim 1, wherein imaging the tissue site comprises performing a PET scan of the tissue site.

11. The method of claim 1, further comprising collecting microbiome data associated with the tissue site.

12. The method of claim 11, wherein collecting microbiome data comprises collecting a biological sample.

13. The method of claim 12, wherein collecting microbiome data further comprises amplifying genetic material from the biological sample.

14. The method of claim 13, wherein collecting the microbiome data further comprises evaluating the genetic material from the biological sample.

15. The method of claim 11, further comprising selecting at least one parameter of a customized splint based upon the microbiome data.

16. The method of claim 1, wherein fabricating the customized tissue support comprises 3-D printing.

17. The method of claim 1, wherein fabricating the customized tissue support comprises 3-D printing a mold.

18. The method of claim 17, wherein fabricating the customized tissue support comprises injection-molding using the mold.

19. A system for providing a customized therapeutic support for therapy of a tissue site, the system comprising:
a customized therapeutic support modeling component configured to:
receive three-dimensional (3-D) data associated with the tissue site; and
select at least one parameter of a customized tissue support based upon the 3-D data; and
a fabrication unit in signal communication with the customized therapeutic support modeling component and configured to fabricate the customized tissue support including the design parameter.

20. The system of claim 19, wherein the customized therapeutic support modeling component is further configured to receive microbiome data associated with the tissue site and to select at least one parameter of a customized tissue support based upon the microbiome data.
